(19) United States
(12) Patent Application Publication
(10) Pub. No.: US 2011/0212143 A1
(43) Pub. Date: Sep. 1, 2011

(54) Ultraviolet Tissue

(76) Inventor: Jung Keun YOON, Cerritoe, CA (US)

(21) Appl. No.: 12/714,272

(22) Filed: Feb. 26, 2010

(51) Int. Cl. A61K 8/02; A61Q 17/04; B65D 77/22

(52) U.S. Cl. 424/401; 424/59; 206/233; 206/823

(57) ABSTRACT

A A ultraviolet tissue comprising: one or more layers of tissue portions stacked on top of each other; water of a unit weight; ethylhexyl methoxycinnamate; butylene glycol; isoamyl p-methoxycinnamate, and fragrance. The mixture of the water, the ethylhexyl methoxycinnamate,the butylene glycol, and the isoamyl p-methoxycinnamate are embedded in the one or more layers of tissue portions. The ultraviolet tissue is configured to produce at least 20 of sun protection factor (SPF) on the skin. The water is a solvent for the other ingredients.

### BACKGROUND OF THE INVENTION

The present invention relates to a ultraviolet tissue. More particularly, this invention relates to a ultraviolet tissue, which contains sun-block liquid.

A sun-block lotion is widely used in the world, especially in countries having a hot and sunny climate.

Usually, the sun-block lotion is packaged in a bottle-type container, carried around, and used directly from the container.

Those types of sun-block lotions are inconvenient due to its bulkiness. They are too large to fit in a small handbag or wallet. And, usage of small amount of them is not facilitated, either.

Accordingly, a need for a ultraviolet tissue has been present for a long time considering the expansive demands in the everyday life. This invention is directed to solve these problems and satisfy the long-felt need.

### SUMMARY OF THE INVENTION

The present invention contrives to solve the disadvantages of the prior art.

An object of the invention is to provide a ultraviolet tissue.

Another object of the invention is to provide a ultraviolet tissue, which contains a sun-block liquid.

Still another object of the invention is to provide a ultraviolet tissue, which combines functions of tissue and sun-block lotion.

An aspect of the invention provides ultraviolet tissue.

A ultraviolet tissue comprising:

one or more layers of tissue portions stacked on top of each other;

water of a unit weight;

ethylhexyl methoxycinnamate of an amount from about 8.0 weight % to about 12.0 weight % relative to the unit weight of water;

butylene glycol of an amount from about 5.0 weight % to about 9.0 weight % relative to the unit weight of water; and

isoamyl p-methoxycinnamate of an amount from about 3.0 weight % to 7.0 weight % relative to the unit weight of water.

Mixture of the water, the ethylhexyl methoxycinnamate, the butylene glycol, and the isoamyl p-methoxycinnamate are embedded in the one or more layers of tissue portions.

The ultraviolet tissue is configured to produce at least 20 of sun protection factor (SPF) on the skin.

The water may be a solvent for the other ingredients.

The mixture of the water, the ethylhexyl methoxycinnamate, the butylene glycol, and the isoamyl p-methoxycinnamate may be configured for blocking sun light incident on a skin of a user.

The mixture may be configured to be applied to the skin while rubbing.

The ultraviolet tissue may further comprise an airtight container for storing the one or more tissue portions and the mixture hermetically.

The airtight container may comprise a perforated line portion for opening the airtight container.

The airtight container may comprise a resealable opening slit.

The ultraviolet tissue may further comprise:

cyclopentasiloxane of an amount from about 1.0 weight % to about 5.0 weight % relative to the unit weight of water;

cyclohexasiloxane of an amount from about 1.0 weight % to about 5.0 weight % relative to the unit weight of water;

zinc oxide of an amount from about 0.5 weight % to about 4.5 weight % relative to the unit weight of water;

titanium dioxide of an amount from about 0.0 weight % to about 4.0 weight % relative to the unit weight of water;

mangifera indica (Mango) seed butter of an amount from about 0.0 weight % to about 3.5 weight % relative to the unit weight of water;

C14-22 alcohol of an amount from about 0.0 weight % to about 3.2 weight % relative to the unit weight of water;

C12-20 alcohol of an amount from about 0.0 weight % to about 3.0 weight % relative to the unit weight of water; and

glyceryl stearate of an amount from about 0.0 weight % to about 3.0 weight % relative to the unit weight of water.

The ultraviolet tissue may further comprise:

cyclopentasiloxane of about 3.0 weight % relative to the unit weight of water;

cyclohexasiloxane of about 3.0 weight % relative to the unit weight of water;

zinc oxide of about 2.5 weight % relative to the unit weight of water;

titanium dioxide of about 2.0 weight % relative to the unit weight of water;

mangifera indica (mango) seed butter of about 1.5 weight % relative to the unit weight of water;

C14-22 alcohol of about 1.2 weight % relative to the unit weight of water;

C12-20 alcohol of about 1.0 weight % relative to the unit weight of water; and

glyceryl stearate of about 1.0 weight % relative to the unit weight of water.

The ultraviolet tissue may further comprise:

PEG-100 stearate of an amount from about 0.6 to about 1.0 weight % relative to the unit weight of water;

cucumis sativus (cucumber) fruit extract of an amount from about 0.3 to about 0.7 weight % relative to the unit weight of water;

rosa centifolia flower water of an amount from about 0.3 weight % to about 0.7 weight % relative to the unit weight of water;

aloe barbadensis leaf juice of an amount from about 0.3 weight % to about 0.7 weight % relative to the unit weight of water;

camellia sinensis leaf extract of an amount from about 0.3 weight % to about 0.7 weight % relative to the unit weight of water;

sodium hyaluronate of an amount from about 0.3 weight % to about 0.7 weight % relative to the unit weight of water; and

lithium magnesium sodium silicate of an amount from about 0.2 weight % to about 0.6 weight % relative to the unit weight of water.

The ultraviolet tissue may further comprise:

PEG-100 stearate of about 0.8 weight % relative to the unit weight of water;

cucumis sativus (cucumber) fruit extract of about 0.5 weight % relative to the unit weight of water;

rosa centifolia flower water of from about 0.3 weight % to about 0.7 weight % relative to the unit weight of water;

aloe barbadensis leaf juice of about 0.5 weight % relative to the unit weight of water;

camellia sinensis leaf extract of about 0.5 weight % relative to the unit weight of water;

sodium hyaluronate of about 0.5 weight % relative to the unit weight of water; and

lithium magnesium sodium silicate of about 0.4 weight % relative to the unit weight of water.

The ultraviolet tissue may further comprise:

tocopheryl acetate of an amount from about 0.1 weight % to about 0.3 weight % relative to the unit weight of water;

allantoin of an amount from about 0.1 weight % to about 0.3 weight % relative to the unit weight of water;

EDTA-2NA of an amount from about 0.1 weight % to about 0.3 weight % relative to the unit weight of water;

methylparaben of an amount from about 0.1 weight % to about 0.3 weight % relative to the unit weight of water;

propylparaben of an amount from about 0.0 weight % to about 0.2 weight % relative to the unit weight of water; and

chlorphenesin of an amount from about 0.05 weight % to about 0.25 weight % relative to the unit weight of water.

The ultraviolet tissue may further comprise:

tocopheryl acetate of about 0.2 weight % relative to the unit weight of water;

allantoin of about 0.2 weight % relative to the unit weight of water;

EDTA-2NA of about 0.2 weight % relative to the unit weight of water;

methylparaben of about 0.2 weight % relative to the unit weight of water;

propylparaben of about 0.1 weight % relative to the unit weight of water; and

chlorphenesin of about 0.15 weight % relative to the unit weight of water.

The ultraviolet tissue may further comprise fragrance of about 0.10 weight % relative to the unit weight of water.

The advantages of the present invention are: (1) the ultraviolet tissue is the convenience, enabling carrying a sun-block lotion thins around; and (2) the ultraviolet tissue functions as a tissue and as a sun-block liquid.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view showing a ultraviolet tissue according to an embodiment of the present invention;

FIG. 2 is a cross-sectional view along line II-II in FIG. 1; and

FIG. 3 is a table showing ingredients of the ultraviolet tissue according to embodiments of the invention.

### DETAILED DESCRIPTION EMBODIMENTS OF THE INVENTION

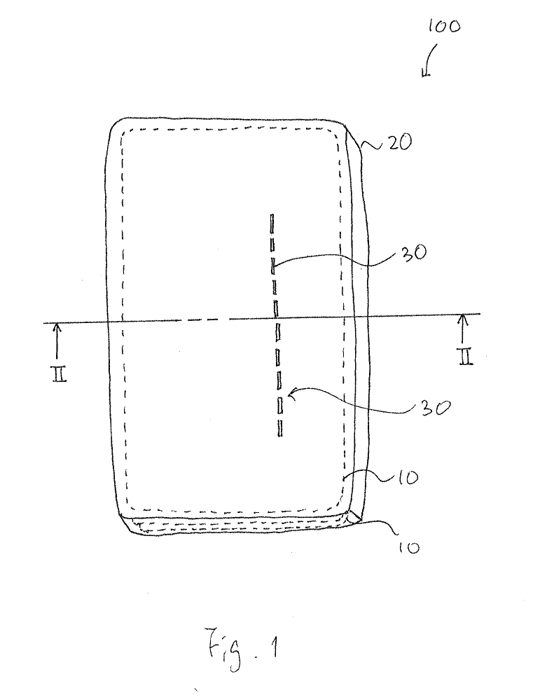

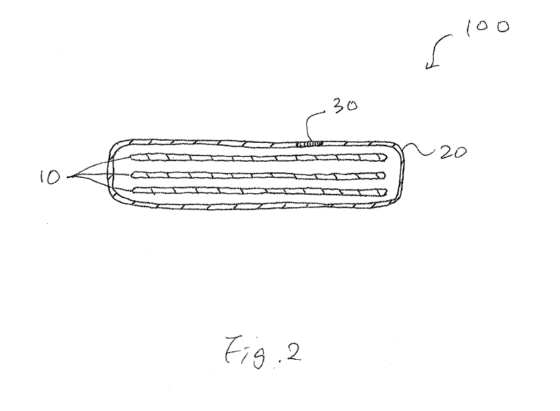

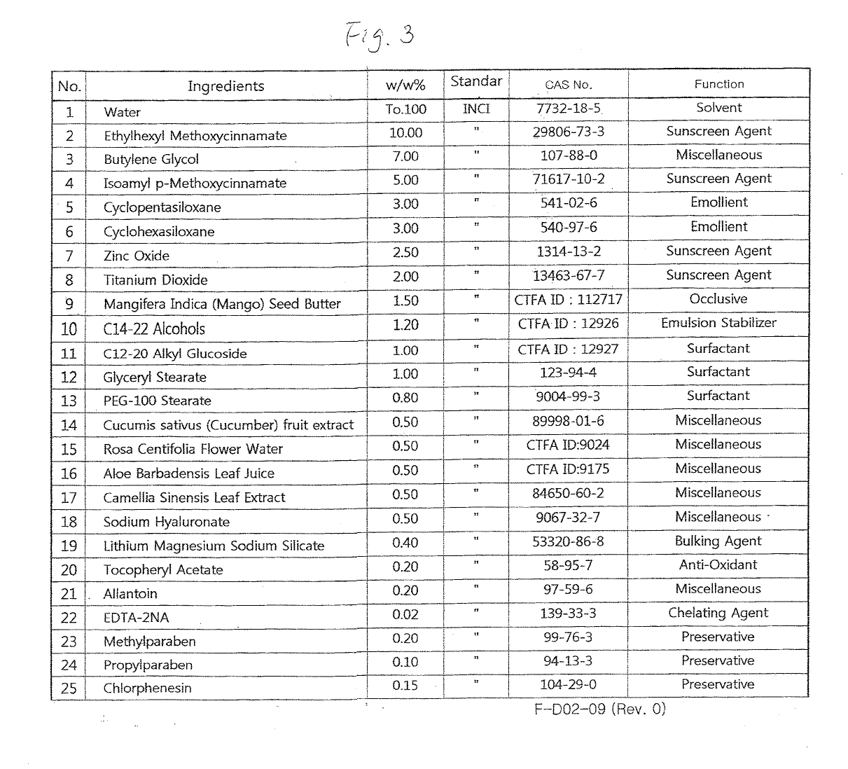

FIGS. 1-3 show a ultraviolet tissue 100 according to an embodiment of the present invention.

An aspect of the invention provides ultraviolet tissue.

A ultraviolet tissue comprising:

one or more layers of tissue portions 10 stacked on top of each other;

water of a unit weight;

ethylhexyl methoxycinnamate of an amount from about 8.0 weight % to about 12.0 weight % relative to the unit weight of water;

butylene glycol of an amount from about 5.0 weight % to about 9.0 weight % relative to the unit weight of water; and

isoamyl p-methoxycinnamate of an amount from about 3.0 weight % to 7.0 weight % relative to the unit weight of water.

Mixture of the water, the ethylhexyl methoxycinnamate, the butylene glycol, and the isoamyl p-methoxycinnamate are embedded in the one or more layers of tissue portions.

The ultraviolet tissue 100 is configured to produce at least 20 of sun protection factor (SPF) on the skin.

The water may be a solvent for the other ingredients.

The mixture of the water, the ethylhexyl methoxycinnamate, the butylene glycol, and the isoamyl p-methoxycinnamate may be configured for blocking sun light incident on a skin of a user.

The mixture may be configured to be applied to the skin while rubbing.

The ultraviolet tissue 100 may further comprise an airtight container 20 for storing the one or more tissue portions 10 and the mixture hermetically.

The airtight container 20 may comprise a perforated line portion 30 for opening the airtight container 20.

Alternatively, the airtight container 20 may comprise a resealable opening slit 30.

The ultraviolet tissue 100 may further comprise:

cyclopentasiloxane of an amount from about 1.0 weight % to about 5.0 weight % relative to the unit weight of water;

cyclohexasiloxane of an amount from about 1.0 weight % to about 5.0 weight % relative to the unit weight of water;

zinc oxide of an amount from about 0.5 weight % to about 4.5 weight % relative to the unit weight of water;

titanium dioxide of an amount from about 0.0 weight % to about 4.0 weight % relative to the unit weight of water;

mangifera indica (Mango) seed butter of an amount from about 0.0 weight % to about 3.5 weight % relative to the unit weight of water;

C14-22 alcohol of an amount from about 0.0 weight % to about 3.2 weight % relative to the unit weight of water;

C12-20 alcohol of an amount from about 0.0 weight % to about 3.0 weight % relative to the unit weight of water; and

glyceryl stearate of an amount from about 0.0 weight % to about 3.0 weight % relative to the unit weight of water.

Alternatively, the ultraviolet tissue 100 may further comprise:

cyclopentasiloxane of about 3.0 weight % relative to the unit weight of water;

cyclohexasiloxane of about 3.0 weight % relative to the unit weight of water;

zinc oxide of about 2.5 weight % relative to the unit weight of water;

titanium dioxide of about 2.0 weight % relative to the unit weight of water;

mangifera indica (mango) seed butter of about 1.5 weight % relative to the unit weight of water;

C14-22 alcohol of about 1.2 weight % relative to the unit weight of water;

C12-20 alcohol of about 1.0 weight % relative to the unit weight of water; and

glyceryl stearate of about 1.0 weight % relative to the unit weight of water.

The ultraviolet tissue 100 may further comprise:

PEG-100 stearate of an amount from about 0.6 to about 1.0 weight % relative to the unit weight of water;

cucumis sativus (cucumber) fruit extract of an amount from about 0.3 to about 0.7 weight % relative to the unit weight of water;

rosa centifolia flower water of an amount from about 0.3 weight % to about 0.7 weight % relative to the unit weight of water;

aloe barbadensis leaf juice of an amount from about 0.3 weight % to about 0.7 weight % relative to the unit weight of water;

camellia sinensis leaf extract of an amount from about 0.3 weight % to about 0.7 weight % relative to the unit weight of water;

sodium hyaluronate of an amount from about 0.3 weight % to about 0.7 weight % relative to the unit weight of water; and

lithium magnesium sodium silicate of an amount from about 0.2 weight % to about 0.6 weight % relative to the unit weight of water.

Alternatively, the ultraviolet tissue 100 may further comprise:

PEG-100 stearate of about 0.8 weight % relative to the unit weight of water;

cucumis sativus (cucumber) fruit extract of about 0.5 weight % relative to the unit weight of water;

rosa centifolia flower water of from about 0.3 weight % to about 0.7 weight % relative to the unit weight of water;

aloe barbadensis leaf juice of about 0.5 weight % relative to the unit weight of water;

camellia sinensis leaf extract of about 0.5 weight % relative to the unit weight of water;

sodium hyaluronate of about 0.5 weight % relative to the unit weight of water; and

lithium magnesium sodium silicate of about 0.4 weight % relative to the unit weight of water.

The ultraviolet tissue 100 may further comprise:

tocopheryl acetate of an amount from about 0.1 weight % to about 0.3 weight % relative to the unit weight of water;

allantoin of an amount from about 0.1 weight % to about 0.3 weight % relative to the unit weight of water;

EDTA-2NA of an amount from about 0.1 weight % to about 0.3 weight % relative to the unit weight of water;

methylparaben of an amount from about 0.1 weight % to about 0.3 weight % relative to the unit weight of water;

propylparaben of an amount from about 0.0 weight % to about 0.2 weight % relative to the unit weight of water; and

chlorphenesin of an amount from about 0.05 weight % to about 0.25 weight % relative to the unit weight of water.

Alternatively, the ultraviolet tissue 100 may further comprise:

tocopheryl acetate of about 0.2 weight % relative to the unit weight of water;

allantoin of about 0.2 weight % relative to the unit weight of water;

EDTA-2NA of about 0.2 weight % relative to the unit weight of water;

methylparaben of about 0.2 weight % relative to the unit weight of water;

propylparaben of about 0.1 weight % relative to the unit weight of water; and

chlorphenesin of about 0.15 weight % relative to the unit weight of water.

The ultraviolet tissue 100 may further comprise fragrance of about 0.10 weight % relative to the unit weight of water.

The ultraviolet tissues 100 can be easily carried around by the user such as a golfer since it is light and compact. Also, it can be easily discarded after using, just like a regular tissue.

By just rubbing the tissue on the skin, the user can form a layer of sun-blocking lotion film.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A ultraviolet tissue comprising:
one or more layers of tissue portions stacked on top of each other;
water of a unit weight;
ethylhexyl methoxycinnamate of an amount from about 8.0 weight % to about 12.0 weight % relative to the unit weight of water;
butylene glycol of an amount from about 5.0 weight % to about 9.0 weight % relative to the unit weight of water; and
isoamyl p-methoxycinnamate of an amount from about 3.0 weight % to 7.0 weight % relative to the unit weight of water,
wherein mixture of the water, the ethylhexyl methoxycinnamate, the butylene glycol, and the isoamyl p-methoxycinnamate are embedded in the one or more layers of tissue portions,
wherein the ultraviolet tissue is configured to produce at least 20 of sun protection factor (SPF) on the skin.

2. The ultraviolet tissue of claim 1, wherein the water is a solvent for the other ingredients.

3. The ultraviolet tissue of claim 1, wherein the mixture of the water, the ethylhexyl methoxycinnamate, the butylene glycol, and the isoamyl p-methoxycinnamate is configured for blocking sun light incident on a skin of a user.

4. The ultraviolet tissue of claim 2, wherein the mixture is configured to be applied to the skin while rubbing.

5. The ultraviolet tissue of claim 1, further comprising an airtight container for storing the one or more tissue portions and the mixture hermetically.

6. The ultraviolet tissue of claim 5, wherein the airtight container comprises a perforated line portion for opening the airtight container.

7. The ultraviolet tissue of claim 5, wherein the airtight container comprises a resealable opening slit.

8. The ultraviolet tissue of claim 1, further comprising:
cyclopentasiloxane of an amount from about 1.0 weight % to about 5.0 weight % relative to the unit weight of water;
cyclohexasiloxane of an amount from about 1.0 weight % to about 5.0 weight % relative to the unit weight of water;
zinc oxide of an amount from about 0.5 weight % to about 4.5 weight % relative to the unit weight of water;
titanium dioxide of an amount from about 0.0 weight % to about 4.0 weight % relative to the unit weight of water;
mangifera indica (Mango) seed butter of an amount from about 0.0 weight % to about 3.5 weight % relative to the unit weight of water;
C14-22 alcohol of an amount from about 0.0 weight % to about 3.2 weight % relative to the unit weight of water;
C12-20 alcohol of an amount from about 0.0 weight % to about 3.0 weight % relative to the unit weight of water; and
glyceryl stearate of an amount from about 0.0 weight % to about 3.0 weight % relative to the unit weight of water.

9. The ultraviolet tissue of claim 1, further comprising:
cyclopentasiloxane of about 3.0 weight % relative to the unit weight of water;
cyclohexasiloxane of about 3.0 weight % relative to the unit weight of water;
zinc oxide of about 2.5 weight % relative to the unit weight of water;
titanium dioxide of about 2.0 weight % relative to the unit weight of water;
mangifera indica (mango) seed butter of about 1.5 weight % relative to the unit weight of water;
C14-22 alcohol of about 1.2 weight % relative to the unit weight of water;
C12-20 alcohol of about 1.0 weight % relative to the unit weight of water; and
glyceryl stearate of about 1.0 weight % relative to the unit weight of water.

10. The ultraviolet tissue of claim 9, further comprising:
PEG-100 stearate of an amount from about 0.6 to about 1.0 weight % relative to the unit weight of water;
cucumis sativus (cucumber) fruit extract of an amount from about 0.3 to about 0.7 weight % relative to the unit weight of water;
rosa centifolia flower water of an amount from about 0.3 weight % to about 0.7 weight % relative to the unit weight of water;
aloe barbadensis leaf juice of an amount from about 0.3 weight % to about 0.7 weight % relative to the unit weight of water;
camellia sinensis leaf extract of an amount from about 0.3 weight % to about 0.7 weight % relative to the unit weight of water;
sodium hyaluronate of an amount from about 0.3 weight % to about 0.7 weight % relative to the unit weight of water; and
lithium magnesium sodium silicate of an amount from about 0.2 weight % to about 0.6 weight % relative to the unit weight of water.

11. The ultraviolet tissue of claim 9, further comprising:
PEG-100 stearate of about 0.8 weight % relative to the unit weight of water;
cucumis sativus (cucumber) fruit extract of about 0.5 weight % relative to the unit weight of water;
rosa centifolia flower water of from about 0.3 weight % to about 0.7 weight % relative to the unit weight of water;
aloe barbadensis leaf juice of about 0.5 weight % relative to the unit weight of water;
camellia sinensis leaf extract of about 0.5 weight % relative to the unit weight of water;
sodium hyaluronate of about 0.5 weight % relative to the unit weight of water; and
lithium magnesium sodium silicate of about 0.4 weight % relative to the unit weight of water.

12. The ultraviolet tissue of claim 11, further comprising:
tocopheryl acetate of an amount from about 0.1 weight % to about 0.3 weight % relative to the unit weight of water;
allantoin of an amount from about 0.1 weight % to about 0.3 weight % relative to the unit weight of water;
EDTA-2NA of an amount from about 0.1 weight % to about 0.3 weight % relative to the unit weight of water;
methylparaben of an amount from about 0.1 weight % to about 0.3 weight % relative to the unit weight of water;
propylparaben of an amount from about 0.0 weight % to about 0.2 weight % relative to the unit weight of water; and
chlorphenesin of an amount from about 0.05 weight % to about 0.25 weight % relative to the unit weight of water.

13. The ultraviolet tissue of claim 11, further comprising:
tocopheryl acetate of about 0.2 weight % relative to the unit weight of water;
allantoin of about 0.2 weight % relative to the unit weight of water;
EDTA-2NA of about 0.2 weight % relative to the unit weight of water;
methylparaben of about 0.2 weight % relative to the unit weight of water;
propylparaben of about 0.1 weight % relative to the unit weight of water; and
chlorphenesin of about 0.15 weight % relative to the unit weight of water.

14. The ultraviolet tissue of claim 1, further comprising fragrance of about 0.10 weight % relative to the unit weight of water.
